(19) United States
(12) Patent Application Publication
(10) Pub. No.: US 2002/0168407 A1
(43) Pub. Date: Nov. 14, 2002

(54) Process for preparing low-viscosity, aqueous cellulose ether solutions

(76) Inventors: Wolfgang Koch, Bomlitz (DE); Peter Wolbers, Walsrode (DE); Bernd Ondruschka, Leipzing (DE); Matthias Nuchter, Leipzig (DE)

Correspondence Address: BAYER CORPORATION, PATENT DEPARTMENT, 100 BAYER ROAD, PITTSBURGH, PA, 15205, US

(21) Appl. No.: 10/114,345

(22) Filed: Apr. 2, 2002

(30) Foreign Application Priority Data
Apr. 6, 2001 (DE) 10117208.7

(51) Int. Cl. A61K 9/36; C07B 31/00

(52) U.S. Cl. 424/480; 204/157.62

(57) ABSTRACT

A process of preparing low-viscosity aqueous cellulose ether solutions is described. The process comprises: (a) dissolving high molecular weight cellulose ether in water to form a first solution; (b) admixing a catalytic amount of an acid into the first solution of step (a) to form a second solution; (c) subjecting the second solution of step (b) to high-frequency radiation having a frequency of 3 to 300,000 MHz, which results in raising the temperature of said second solution to a temperature of 60° C. to 150° C.; (d) cooling the frequency subjected second solution of step (c); and (e) neutralizing the cooled solution. Also described is a method of using the cellulose ether prepared in accordance with the method of the present invention as a coating material for solid pharmaceutical administration units, such as tablets.

### CROSS REFERENCE TO RELATED PATENT APPLICATION

The present patent application claims the right of priority under 35 U.S.C. §119 (a)-(d) of German Patent Application No. 101 17 208.7, filed Apr. 6, 2001.

### FIELD OF THE INVENTION

The invention relates to a process for preparing low-viscosity, aqueous cellulose ether solutions, in which relatively high molecular weight cellulose ethers are dissolved in water and admixed with catalytic amounts of acid. The viscosity of the aqueous cellulose ether solutions is reduced and the solutions are subsequently neutralized again in accordance with the acid added. Cellulose ethers which are obtainable from these low-viscosity cellulose ether solutions are used, inter alia, for the production of pharmaceutical administration units, for example tablets. In addition, low molecular weight cellulose ethers such as carboxymethylcellulose (CMC), methylcellulose (MC), methylhydroxyalkylcelluloses (e.g. MHPC or MHEC) and hydroxyethylcellulose (HEC), are used as additives in many applications.

### BACKGROUND OF THE INVENTION

Cellulose ethers are usually obtained by alkalization of cellulose and subsequent reaction with alkyl or aryl halides or by reaction of the alkali metal cellulose with alkyl or aryl epoxides. The reactions of alkali metal celluloses with methyl chloride, ethyl chloride, ethylene oxide, propylene oxide or 1,2-epoxybutane are particularly well known.

Various methods of obtaining low-viscosity cellulose ether solutions from long-chain cellulose derivatives have been described, for example irradiation with electrons (DE 4 434 280 A1), treatment with enzymes (e.g., EP 382,576) or hydrogen peroxide (e.g., U.S. Pat. No. 3,728,331 and EP 465,992) or the use of acids (e.g., U.S. Pat. No. 3,497,496). Molecular weight degradation using acidic gases such as hydrogen chloride is, in particular, subject-matter of many patent applications (e.g., U.S. Pat. No. 3,391,135 and U.S. Pat. No. 4,061,859).

U.S. Pat. No. 5,196,069 describes the molecular weight degradation of cellulose by degrading cellulose by means of enzymes to form low molecular weight carbohydrates. For this molecular weight degradation using the said enzymes, the cellulose is pretreated with the assistance of microwaves in an acidic, aqueous solution, in particular a solution acidified with acetic acid. Even if it is not explicitly stated, it may be assumed that the molecular weight degradation once again occurs as a result of the enzyme treatment following the microwave pretreatment.

From an industrial point of view, it is desirable to produce low-viscosity cellulose ether solutions in a manner which is quick and simple in process engineering terms and is also inexpensive. The use of small amounts of acid and very short reaction times with little energy consumption is particularly desirable. The need for particularly low amounts of impurities in products, in particular for the pharmaceuticals sector, allows the use of only small amounts of reagents which catalyze the molecular weight degradation. Furthermore, a continuous process is particularly desirable to achieve a high space-time yield.

### SUMMARY OF THE INVENTION

It is therefore an object of the present invention to provide a process for preparing low-viscosity, aqueous cellulose ether solutions which requires very short reaction times at a low energy consumption and only a minimum of additional chemicals for the molecular weight degradation.

According to the invention, the molecular weight degradation of relatively high-viscosity cellulose ether solutions is not carried out as described in the background by means of enzymes, hydrogen peroxide, acidic gases or other methods as mentioned above. Instead, in accordance with the present invention, there is provided a process of preparing low-viscosity aqueous cellulose ether solutions comprising:

(a) dissolving high molecular weight cellulose ether in water to form a first solution;

(b) admixing a catalytic amount of an acid into the first solution of step (a) to form a second solution;

(c) subjecting the second solution of step (b) to high-frequency radiation having a frequency of 3 to 300,000 MHz, which results in raising the temperature of said second solution to a temperature of 60° C. to 150° C.;

(d) cooling the frequency subjected second solution of step (c), e.g., to a temperature of less than 60° C., such as a temperature of from 25° C. to 35° C.; and

(e) neutralizing the cooled solution.

In the method of the present invention, steps (a) and (b) may be performed concurrently. In addition, the cellulose ether may be isolated from the neutralised solution of step (e) in accordance with methods known to the skilled artisan.

Unless otherwise indicated, all numbers expressing quantities of ingredients, reaction conditions, etc. used in the specification and claims are to be under stood as modified in all instance by the term “about.”

### DETAILED DESCRIPTION OF THE INVENTION

The process claimed according to the invention makes it possible to prepare low-viscosity cellulose ether solutions in surprisingly short reaction times. Thus, when using catalytic amounts of acid (e.g., 0.01-1% by weight based on cellulose ether) combined with treatment with high-frequency radiation in the frequency range of 3 to 300,000 MHz (e.g., from 10 to 10,000 MHz) and the resultant heating of the reaction solution in the temperature range T=60-150° C. allows for reaction times of 0.5 -60 minutes, in particular 0.5-30 minutes and even 0.5-15 minutes, to be achieved.

Furthermore, the high frequency treatment ensures an acceptable energy consumption with the introduction of energy being able to be carried out either in pulses or continuously. The high frequency power to be set for the respective reaction is determined by the industrial application and its parameters (throughput, apparatus geometry).

As a result of the molecular weight degradation effected by this high frequency treatment, no particular additional reagents, e.g. enzymes, are required for the preparation of the low-viscosity cellulose ether solutions, unlike the case of known processes. Consequently, only minimal amounts of by-products are formed. Only catalytic amounts of acid are necessary. The cellulose ethers which can be obtained from the cellulose ether solutions are thus particularly suitable for use in pharmaceutical related applications where it is critical that products contain extremely small amounts of impurities, for example for the production of coating materials for solid pharmaceutical administration units, such as tablets.

Cellulose ethers are derivatives of cellulose which are typically prepared by replacement of the hydrogen of the alcohol groups with alkyl groups, aryl groups or hydroxyalkyl groups. In addition, the alkyl groups can also be substituted by other groups, e.g. ionic alkylammonium groups. The cellulose ethers, which can also be mixed ethers having different substituent groups, are dissolved in water in concentrations of 0.1-20% by weight. Preference is given to using concentrations of 1-15% by weight, in particular concentrations of 8-12% by weight, of cellulose ethers in distilled water.

Cellulose ethers that may be used in the process of the present invention include, but are not limited to, methylcellulose, methylhydroxyethylcellulose, methylhydroxypropylcellulose, hydroxyethylcellulose, hydroxypropylcellulose, ethylhydroxyethylcellulose and mixtures thereof. As used herein and in the claims the term “high molecular weight cellulose ether” means cellulose ethers having viscosities, as measured on a solution containing 2% by weight of cellulose ether in distilled water at a shear rate of D=2.55 s⁻¹ and at a temperature of 20° C. (i.e., a V₂ viscosity), of at least 400 mPas, e.g., at least 15,000 mPas, and preferably from 15,000 mPas to 80,000 mPas. The viscosities are typically determined using a suitable rotational viscometer, e.g., from Haake.

As acid, either an organic acid such as acetic acid or formic acid or an inorganic (or mineral) acid such as hydrochloric acid, nitric acid, phosphoric acid, hydrofluoric acid or sulphuric acid is added. Preference is given to using hydrochloric acid or sulphuric acid. The acid is, as mentioned above, used in catalytic amounts of 0.01-1% by weight, based on the cellulose ether, preferably in an amount of 0.05-0.8% by weight, in particular 0.1-0.7% by weight, in each case based on the cellulose ether.

The high frequency treatment according to the invention makes it possible to prepare clear cellulose ether solutions having only slight yellowing (APHA<70 for a 4% strength by weight cellulose ether solution) and low viscosities (V₂=1-100 mPas, preferably V₂=1-30 mPas, in particular V₂=1-20 mPas).

In neutralization step (e) in the process of the present invention, the neutralizing agent may be selected from at least one of an alkali metal carbonate, an alkaline earth metal carbonate, an alkali metal bicarbonate, an alkaline earth metal bicarbonate, an alkali metal hydroxide and an organic amine. In a preferred embodiment of the present invention, the neutralizing agent is selected from at least one of sodium carbonate, sodium bicarbonate, ammonium carbonate, ammonium bicarbonate and ammonia.

The viscosities V are measured on aqueous cellulose ether solutions containing a defined proportion of the cellulose ether of from 1 to 15% by weight in distilled water at a shear rate of D=2.55 s⁻¹ and a temperature of 20° C. (e.g., V₂ for a 2% strength by weight solution). The measurements of the viscosities were carried out using a rotation viscometer from Haake.

The process of the present invention can be carried out either continuously or batchwise in generally available apparatuses.

The present invention is also directed to a method of using the cellulose ether solution formed in the process of claim 1 in the preparation of a coating material for solid pharmaceutical administration units, such as tablets. When used in such applications, the neutralized and cooled solution of step (e) may be used directly, or the cellulose ether may be first isolated therefrom.

The invention is illustrated by the examples below, which are not intended to limit the scope of the invention.

### EXAMPLES

### Example 1

8 parts by weight of methylhydroxypropylcellulose (MHPC, Walocel MK 400 PF, viscosity V₂=about 400 mpas) are homogeneously mixed with 0.65 parts by weight of hydrochloric acid (1 molar) and 391.35 parts by weight of water by stirring for 30 minutes with a magnetic stirrer, so that a colourless solution is formed. The reaction mixture is subsequently treated in a batch procedure at a frequency of 2450 MHz and a power of 500 W in a microwave laboratory system (MLS ETHOS MR) for 30 minutes at 1250C. After the reaction is complete, the reaction mixture is cooled to 35° C. and neutralized by addition of 55 mg of sodium bicarbonate. The MHPC solution obtained in this way has a viscosity of V₂=10 mPas and an APHA value of 10.

### Example 2

40 parts by weight of methylhydroxypropylcellulose (MHPC, Walocel MK 15000 PF, viscosity V₂=about 15000 mPas are homogeneously mixed with 20 parts by weight of hydrochloric acid (0.1 molar) and 338 parts by weight of water, so that a colourless solution is formed. The reaction mixture is subsequently heated to 115° C. using 1000 W of microwave power (duration: about 3 min) and treated in a batch procedure at a frequency of 2450 MHz and a power of 400 W in a microwave laboratory system (MLS ETHOS MR) for 15 minutes at 115° C. The reaction mixture is cooled to 30° C. and neutralized by addition of 2 parts by weight of sodium bicarbonate solution (1 molar). The MHPC solution obtained in this way has a viscosity of V₁₀=113 mpas. The APHA value of the corresponding 4% strength by weight MHPC solution is 50.

Note: A 4% strength by weight MHPC solution prepared by means of thermal heating and having a viscosity of V₁₀=about 100 mPas has an APHA value of>150.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process of preparing low-viscosity aqueous cellulose ether solutions comprising:
(a) dissolving high molecular weight cellulose ether in water to form a first solution;
(b) admixing a catalytic amount of an acid into the first solution of step (a) to form a second solution;
(c) subjecting the second solution of step (b) to high-frequency radiation having a frequency of 3 to 300,000 MHz, which results in raising the temperature of said second solution to a temperature of 60° C. to 150° C.;
(d) cooling the frequency subjected second solution of step (c); and
(e) neutralizing the cooled solution.

2. The process of claim 1 wherein the high frequency radiation has a frequency of 10 to 10,000 MHz.

3. The process of claim 1 wherein said second solution is subjected to high frequency radiation for a period of 0.5 to 60 minutes.

4. The process of claim 1 wherein the second solution is subjected to high-frequency radiation in pulses.

5. The process of claim 1 wherein the second solution is continuously subjected to high-frequency radiation.

6. The process of claim 1 wherein the cellulose ether is selected from the group consisting of methylcellulose, methylhydroxyethylcellulose, methylhydroxypropylcellulose, hydroxyethylcellulose, hydroxypropylcellulose and ethylhydroxyethylcellulose.

7. The process of claim 1 wherein said acid is selected from one of organic acids and inorganic acids.

8. The process of claim 7 wherein said acid is selected from one of hydrochloric acid and sulfuric acid.

9. The process of claim 7 wherein said acid is present in a catalytic amount of 0.01 to 1 percent by weight, based on the weight of cellulose ether.

10. The process of claim 1 wherein said first solution has a cellulose ether content of 0.1 to 20 percent by weight.

11. The process of claim 1 wherein the cooled solution is neutralized in step (d) with a member selected from at least one of an alkali metal carbonate, an alkaline earth metal carbonate, an alkali metal bicarbonate, an alkaline earth metal bicarbonate, an alkali metal hydroxide and an organic amine.

12. The process of claim 11 wherein said member is selected from at least one of sodium carbonate, sodium bicarbonate, ammonium carbonate, ammonium bicarbonate and ammonia.

13. The process of claim 1 wherein the process is conducted as a continuous process.

14. The process of claim 1 wherein the process is conducted as a batch process.

15. A method of using the cellulose ether formed in the process of claim 1 in the preparation of a coating material for solid pharmaceutical administration units.
